(19) United States
(12) Patent Application Publication
(10) Pub. No.: US 2013/0344114 A1
(43) Pub. Date: Dec. 26, 2013

(54) COMPOSITION FOR REPAIRING CARTILAGE TISSUE AND METHOD FOR MAKING THE SAME

(71) Applicants: Nai-Jen Chang, Tainan City (TW); Ming-Long Yeh, Tainan City (TW); Chih-Chan Lin, Tainan City (TW)

(72) Inventors: Nai-Jen Chang, Tainan City (TW); Ming-Long Yeh, Tainan City (TW); Chih-Chan Lin, Tainan City (TW)

(21) Appl. No.: 13/607,397

(22) Filed: Sep. 7, 2012

(30) Foreign Application Priority Data
Jun. 22, 2012 (TW) 101122501

(51) Int. Cl. A61K 35/12; A61K 9/00; C12N 5/071; A61P 19/04

(52) U.S. Cl. 424/400; 424/93.7; 435/395; 435/396

(57) ABSTRACT

A composition for repairing cartilage tissues includes a scaffold and a plurality of endothelial progenitor cells. The endothelial progenitor cells adhere on the scaffold. A method of making the composition for repairing cartilage tissue is also disclosed. This is advantageous for safely and quickly repairing cartilage tissues by using the composition and the manufacturing method thereof.

### CROSS REFERENCE TO RELATED APPLICATIONS

This Non-provisional application claims priority under 35 U.S.C. §119(a) on Patent Application No(s). 101122501 filed in Taiwan, Republic of China on Jun. 22, 2012, the entire contents of which are hereby incorporated by reference.

### BACKGROUND OF THE INVENTION

1. Field of Invention

The present invention relates to a composition for repairing cartilage tissues and a method of making the same, and more particularly, to a composition containing endothelial progenitor cells and a method making the same.

2. Related Art

Cartilage tissue located at the joint surface of the bones is white and transparent tissue. The cartilage tissue is able to transmit the stress of the bone tissue, absorb the striking force between the bone layer and the joint surface, reduce the friction force of the joint surface and assist the joint in some kind of exercise, like sliding or rolling, with the muscle and ligament tissue. Thus, the cartilage tissue is able to protect the bone tissue in the joint, and further mitigating the wearing of the bone as bearing the foreign stress.

However, the cartilage tissue is a connective tissue without blood vessel, lymphatic system, and nerve. It is mainly composed of hyaline cartilage, type II collagen and proteoglycans. Once the cartilage tissue is damaged, it is hard to be repaired due to the limited amount of the chondrocyte nearby. Even more, the covering of the extracellular matrix makes it hard for the chondrocyte to reach the damaged area.

What currently known is, the repairing reaction will occur as the damage reaching the subchondral bone. However, most of the newly formed tissues are fibrocartilage tissue mainly composed of type I collagen. Because the fibrocartilage tissue lacks the bio-mechanical feature of the cartilage tissue and the function of the hyaline cartilage, it will be gradually degraded. Furthermore, the fibrocartilage tissue is unable to assist the bone recovering to the condition before damage.

The method for repairing the cartilage tissues is different with the level of the damage of the cartilage tissues. Physical therapy, oral medication, steroid are used for patients with minor ailments to ease the pain and swelling of the joint.

For those patients with wearing cartilage tissues, injection of hyaluronic acid and drilling method are used. But for those with severe wearing cartilage tissues, surgery or even arthroplasty is probably a more effective method. However, the lifespan of metal joint is limited. In most of the cases, another arthroplasty or surgery will be needed.

In recent years, the development of tissue engineering, like osteochondral grafting or chondrocyte implantation for cartilage repairing is fast. However, both of the two methods use invasive methods to take the cartilage tissues from other part of the body, and further sending the tissues or cells into the affected area by surgery which cause the other damage to the donating source. That means, patients with damaged cartilage tissues have to experience the pain of surgeries at least twice. Moreover, the defect and degeneration of the donating source or the uneven distribution may occur. In addition, the in-vitro cell culture takes 3 to 4 weeks during the treatment. Patients need to spend a long time waiting and experiencing torture. More importantly, the cells from the above source mostly form fibrocartilage cells mainly composed of type I collagen rather than the hyaline cartilage having type II collagen needed by the joint cartilage. Hence, its repairing effect is limited.

Therefore, it is an important subject to provide a low-invasive and short-time manufacturing method of a composition to form higher ratio of hyaline cartilage in repairing treatment, and further approve the efficacy and the scope of the cartilage repair by applying the tissue engineering.

### SUMMARY OF THE INVENTION

In view of the foregoing, it is an object of the present invention to provide a low-invasive and short-time manufacturing method of a composition to form higher ratio of hyaline cartilage in repairing treatment, and further approve the efficacy and the scope of the cartilage repair by applying the tissue engineering.

To achieve the above, the present invention discloses a composition for repairing cartilage tissues includes a scaffold and a plurality of endothelial cells. The endothelial cells adhere on the scaffold.

In one embodiment of the present invention, the material of scaffold is bio-compatible substance.

In one embodiment of the present invention, the material of the scaffold comprises polycaprolactone (PCL), polylactic acid (PLA), polyglycolic acid (PGA), poly(lactic-co-glycolic) acid (PLGA), poly(p-dioxanone), polyanhydride, polyethylene terephthalate (PET), polyorthoester (POE), collagen, gelatin, hyaluronic acid, chitosan, or polyethylene glycol (PEG).

In one embodiment of the present invention, the scaffold is multi-porous scaffold.

In one embodiment of the present invention, the endothelial progenitor cells are taken from the blood of an individual having cartilage tissues to be repaired.

In one embodiment of the present invention, the composition is an implant.

In one embodiment of the present invention, the composition is implanted into the adjacency between the cartilage tissue and its peripheral bone tissue of an individual.

In one embodiment of the present invention, the endothelial progenitor cells induce the cartilage tissues and its peripheral bone tissue to grow and repair.

To achieve the above, the present invention also discloses a method of making a composition for repairing cartilage tissue comprises the following steps of: providing a scaffold; implanting a plurality of endothelial progenitor cells on the scaffold; and culturing the endothelial progenitor cells on the scaffold.

In one embodiment of the present invention, the scaffold is bio-compatible substance.

In one embodiment of the present invention, the material of the scaffold comprises polycaprolactone (PCL), polylactic acid (PLA), polyglycolic acid (PGA), poly(lactic-co-glycolic) acid (PLGA), poly(p-dioxanone), polyanhydride, polyethylene terephthalate (PET), polyorthoester (POE), collagen, gelatin, hyaluronic acid, chitosan, or polyethylene glycol (PEG).

In one embodiment of the present invention, the scaffold is multi-porous.

In one embodiment of the present invention, the endothelial progenitor cells are taken from the blood of an individual having cartilage tissues to be repaired.

In one embodiment of the present invention, the composition is an implant.

In one embodiment of the present invention, the composition is implanted into the adjacency between the cartilage tissue and its peripheral bone tissue of an individual.

In one embodiment of the present invention, the endothelial progenitor cells induce the cartilage tissues and its peripheral bone tissue to grow and repair.

In one embodiment of the present invention, the endothelial progenitor cells are cultured in vitro within one week before implanting into the scaffold.

In one embodiment of the present invention, the endothelial progenitor cells are cultured on the scaffold within one day.

The word “repair” used here is collectively referred to an action of recovering, maintaining or improving the function of biological tissues by some materials or means. Preferably, repairing means the action to recover, maintain or improve the function of the damaged biological tissues.

As mentioned above, the composition for repairing cartilage tissues provided by the present invention comprises the endothelial progenitor cells and the cell scaffold materials. Because of the simple way to harvest the endothelial progenitor cells by drawing blood, this method is able to ease the burden of invasive surgery and the pain from drawing marrow in the past. In addition, the composition composed of the endothelial progenitor cells is advantageous for short culturing time and the small amount of cell demand. This is able to shorten the treatment of cartilage repairing.

Furthermore, the composition composed of the endothelial cells and the biological scaffold and the method making the same have the following advantages. First, the endothelial progenitor cells are taken from the patient himself to prevent from the infection of the immune rejection in the allograft or the xenograft. Second, the composition is able to induce the cartilage tissues and its peripheral bone tissues to grow and repair; furthermore, forming hyaline cartilage is able to raise the effect of repairing and to recover the whole joint function of patients. Compared to the conventional techniques, the composition and the method for making the same provided by the present invention are advantageous for decreasing the surgery times, shortening the treatment and achieving the efficacies of fast and effectively repairing the cartilage tissues

### BRIEF DESCRIPTION OF THE DRAWINGS

The present invention will become more fully understood from the subsequent detailed description and accompanying drawings, which are given by way of illustration only, and thus are not limitative of the present invention and wherein:

FIG. 1A is a schematic view of a composition according to one embodiment of the present invention;

FIG. 1B is a photo showing the composition according to one embodiment of the present invention;

FIG. 2 is a flow chart showing the steps of the manufacturing method of the composition for repairing cartilage tissues in accordance with an embodiment of the present invention;

FIG. 3 is an experimental result of the adherence of the endothelial progenitor cells onto the PLGA scaffold;

FIG. 4 is a schematic view showing the appearance of the cartilage tissue repaired by the methods of the present invention;

FIG. 5 shows some images of the cartilage tissue repaired by the methods of the present invention observed by micro computed tomography (microCT); and

FIG. 6 is the result of the type II collagen included by the repaired cartilage tissue observed by immuno-histochemistry stain.

### DETAILED DESCRIPTION OF THE INVENTION

The present invention will be apparent from the following detailed description, which proceeds with reference to the accompanying drawings, wherein the same references relate to the same elements.

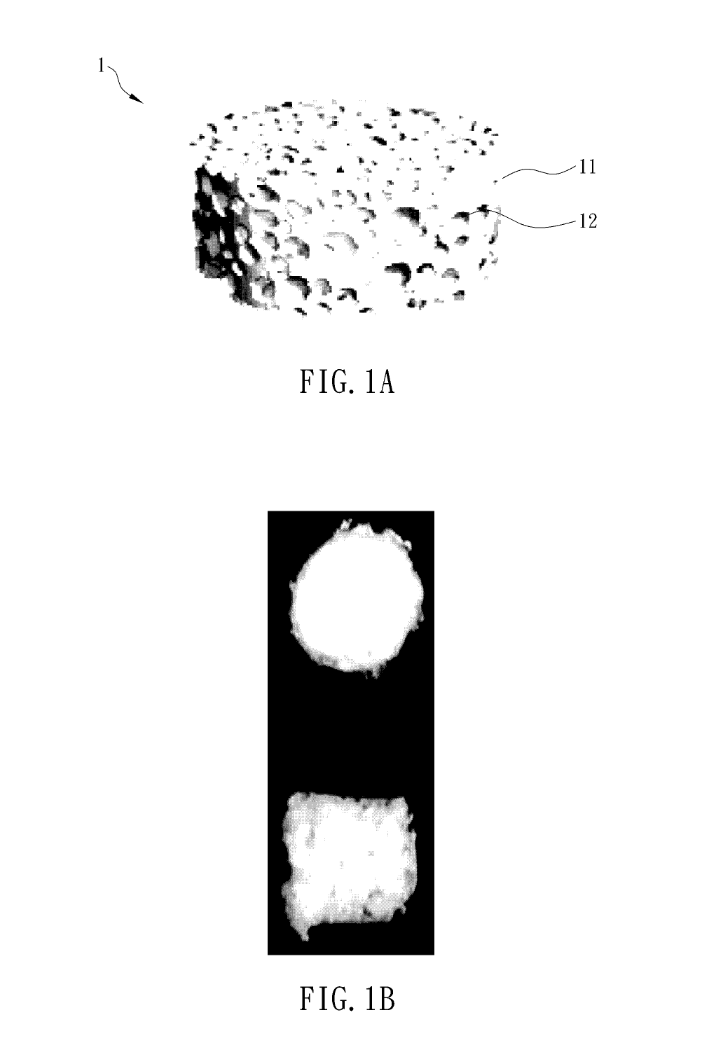

FIG. 1A is a schematic view of a composition according to one embodiment of the present invention. FIG. 1B is a photo showing the composition according to one embodiment of the present invention. The composition drafted in FIG. 1A is for convenient description. The real view of the composition is referred to FIG. 1B. With reference to FIG. 1A and FIG. 1B, the composition 1 for repairing cartilage tissues provided by the present invention includes a scaffold 11 and a plurality of endothelial progenitor cells 12. The endothelial progenitor cells 12 adhere on the scaffold 11. In this embodiment, the cartilage tissue repairing is happened to form new chondrocytes or to fill the damage area of the cartilage tissues under the premise of implantation of the composition and the recovering or improving the damage area of the cartilage tissue. The following is mainly referred to the description of the composition 1.

In this embodiment, the composition 1 is for implantation. Other methods for bringing the composition 1 to achieve the area of the cartilage tissue to be repaired can also be used, and the present invention is not limited to this method. As the composition 1 is being implanted into an individual, it is preferably implanted into the place adjacent to the area to be repaired; that is, to the adjacency between the cartilage tissue and its peripheral bone tissue of an individual. The “individual” used here is preferably referred to an organism. It mainly comprises mammals like mouse, human, rabbit, cow, sheep, pig, monkey, dog and cat, preferably human. And the cartilage tissue is preferably to be human's articular cartilage.

The material of Scaffold 11 can be bio-degradable, bio-absorbable, bio-compatible substance or the combination of any substance mentioned above. More specifically, it includes polycaprolactone (PCL), polylactic acid (PLA), polyglycolic acid (PGA), poly(lactic-co-glycolic) acid (PLGA), poly(p-dioxanone), polyanhydride, polyethylene terephthalate (PET), polyorthoester (POE), collagen, gelatin, hyaluronic acid, chitosan or poly(ethylene glycol) (PEG), but the present invention is not limited to the material. In this embodiment, the material of the scaffold 11 is substantially poly (lactic-co-glycolic) acid (PLGA).

Poly (lactic-co-glycolic) acid (PLGA) is polymerized by poly lactic acid and glycolic acid by different proportion. Practically, the range of the mixing proportion of the poly lactic acid and the glycolic acid is from about 1:1 to about 9:1. More practically, the ratio is 50:50, 60:40, 65:35, 70:30, 75:25, 80:20, 85:15 or 90:10. The more the glycolic acid included in copolymer, the slower the degradation of the copolymer. For example, when the ratio of the poly lactic acid and the glycolic acid is 85:15, the composition 1 containing the formed scaffold 11 can offer better repairing effect.

Otherwise, the scaffold 11 can be coated, cladded or modified by other substances, like growth factors or natural substances, beneficial to the growth of the cells, and the present invention is not limited to this construction. The scaffold 11 is preferably multi-porous scaffold which is also advantageous for the adherence and the growth of the cells.

The endothelial progenitor cells 12 are taken from the blood of an individual which has cartilage tissues to be repaired. Preferably, the cells are taken from the blood of the individual, and then centrifugating for several times to get pure cells.

By using the materials and the proportion mentioned above, the scaffold 11 is well-prepared. The manufacturing method of the scaffold 11 is well-understood by the person having ordinary skill in the art, and are not repeated here. Subsequently, mix the blood with HBSS with the ratio 1:2, and centrifugate with the Ficoll-Hypaque method. Then, extract the mononuclear layer and centrifugate for several times. Culture the residual cells onto the dish coated with fibronectin to get endothelial progenitor cells 12.

Next, inject the solution containing endothelial progenitor cells 12 into the scaffold, and incubate both of the cells and the scaffold. In detail, due to the multi-porous structure of the scaffold, the endothelial progenitor cells 12 can randomly adhere onto the surface of the scaffold 11, the porous structure of the scaffold 11 or the combination of the above, and the present invention is not limited to this construction.

As mentioned above, it takes merely a short time to finish the preparation of the composition 1 after the implantation of the endothelial progenitor cells 12. Compared to the long time the prior techniques takes, the present invention is advantageous for instant application and short-time treatment. Because the composition 1 is injected into the adjacency between the cartilage tissues and its peripheral bone tissues, the composition 1 is able to induce the cartilage tissues and its peripheral bone tissues to grow and move rather than culturing the cells in vitro, and further completing the repairing. Thus, the culturing time of the endothelial progenitor cells 12 on the scaffold 11 is short. Stable adherence is the chief demand. In practical application, the day of culturing can be 1 day, 2 days, 1 week, or 2 weeks, preferably within 1 day. Due to the same reason, the material of the scaffold 11 can be used without the surface modification. For certain, in the better embodiment, the surface of the scaffold 11 can be modified to shorten the time for the endothelial progenitor cells 12 adherence, and the present invention is not limited to the construction.

After forming the composition 1 of the present invention by the methods mentioned above, the implantation can be performed according to the position to be repaired. In detail, in this embodiment, the composition 1 is implanted into the adjacency between the cartilage tissues and the bone tissues. Preferably, the composition 1 is implanted into the adjacency between the hyaline cartilage tissues and its subchondral bone, or the so called damaged osteochondral area. In addition, the word “damaged” used herein is collectively referred to the wearing, softening, crashing or diminishing of the cartilage tissues, and is further causing defect or even splicing of the cartilage tissues. Otherwise, the word “implant” used herein is collectively referred to the action of forming an orifice on the surface of the individual and sending the composition 1 into the preset location. This implantation method is helpful for the medical stuff to precisely place the composition 1 into the correct location and further raise the effect of the repairing. However, the present invention is not limited to the method. In other embodiments, injection method advantageous for saving the time and easing the pain of patients is also available for imputing the composition 1 into the individual.

After implanting into the position to be repaired, the endothelial progenitor cells 12 are able to stay at the implanting location without flowing or leaving by adhering on the scaffold 11. Furthermore, the endothelial progenitor cells induce the cartilage tissues and its peripheral bone tissue to grow and repair. That is, the implantation of the endothelial progenitor cells is able to promote the proliferation, growth and differentiation of the cartilage tissues, and thus generating the hyaline cartilage cells and covering and/or filling the damaged area.

In this embodiment, the endothelial progenitor cells 12 are harvested by drawing blood and purring. That is, there is no invasive action into the organism when the cells are harvested. The individual has only to experience the implantation surgery of the composition 1 once, and thus dispensing the invasive methods, like surgery or marrow drawing, to harvest cells and decreasing the pain and the risk patients may experience.

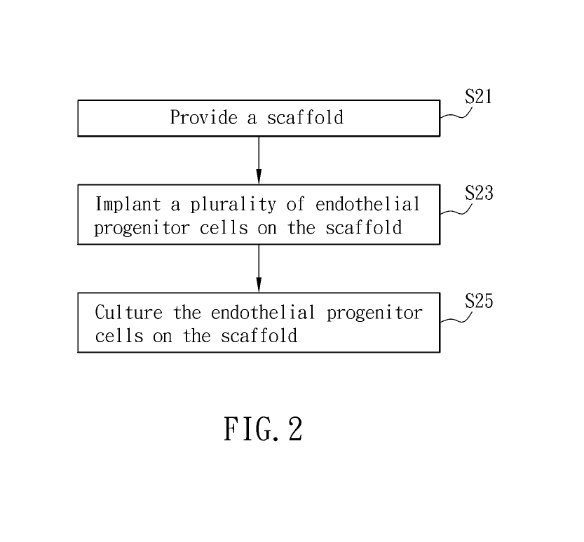

The present invention also provides a manufacturing method for making the composition mentioned above. FIG. 2 is a flow chart of the steps of the manufacturing method of the composition for repairing cartilage tissues in accordance with an embodiment of the present invention. With reference to FIG. 2, in this embodiment, the steps includes providing a scaffold (S21), implanting a plurality of endothelial progenitor cells on the scaffold (S23), and culturing the endothelial progenitor cells on the scaffold (S25). But the techniques and the implementation details of the steps has been disclosed by the above-mentioned description. The details of the steps can also be referred to the following experiment, and are not repeated here. Otherwise, the manufacturing method of the scaffold can be referred to the manufacturing methods of ordinary cell scaffold. And the methods of cell purification method can also be referred to the isolation of mononuclear layer from whole blood. These methods are well-understood by the person having ordinary skill in the art, and are not repeated here.

Particularly, in the manufacturing methods mentioned above, the endothelial progenitor cells are cultured in-vitro for one day, two days, one week or two weeks before implanting into the scaffold. That is, the endothelial progenitor cells harvested from the blood drawn from the organism are able to adhere onto the scaffold with short-time purification and culture. That's because the cells applied by the present invention is used to induce the cartilage tissues and/or their peripheral bone tissues growing, and further completing the repairing. Hence, the amount of the cells used in present invention is small, and the manufacturing time of the composite is effectively shortened.

In addition, same as the description above, in this embodiment, endothelial progenitor cells are able to complete the adherence within one day. Overall, the manufacturing method of the composition can ne completed within seven days. This is advantageous for saving manpower, materials and the treatment time, and accelerating the repairing.

The following and accompanying figures take a number of experiments for examples to describe the manufacturing method of the composition and the practical applying method and the effect of the implantation of the composition in accordance with the embodiments of the present invention.

Experiment 1: the Seeding of Endothelial Progenitor Cells into the PLGA Scaffold

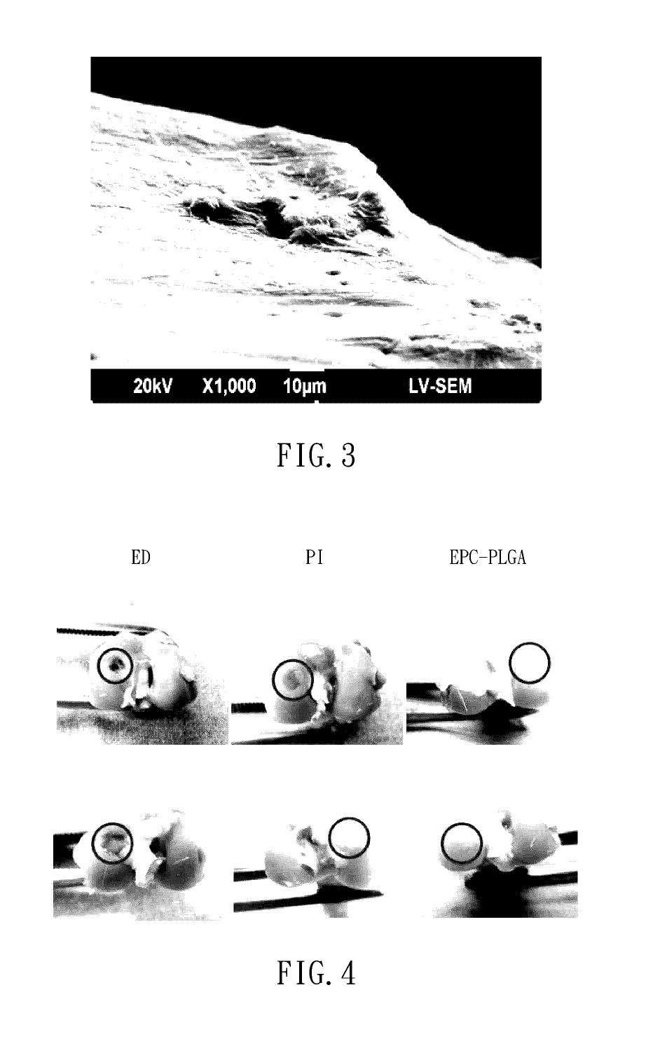

The endothelial progenitor cells were cultured on the dish with 10% trypsin. The PLGA scaffolds were infiltrated in 75% ethanol for 5 minutes. Then, the scaffolds were washed with PBS for 5 times and placed in a 24 well plate and put aside for later usage. The endothelial progenitor cells were cultured in the dish with 10% trypsin. In the meantime, the cells were counted and adjusted the concentration at 5*105 cells/ml. 100 μL of cell solution was injected with syringe. And the solution was checked for fully infiltrating into the scaffold. The dish is incubated in 37° C. for 4 hours. After the 4 hours incubation, 1.5 ml of medium is filled in, and then incubating in 37° C. After 24 hour of cultivation, the scaffold attached with cells is ready for implantation. The result is referred to FIG. 3.

FIG. 3 is an experimental result of the adherence of the endothelial progenitor cells onto the PLGA scaffold. With reference to FIG. 3, where the arrow point is the adhered endothelial progenitor cells on the PLGA scaffolds. This result clearly illustrates that the endothelial progenitor cells and the PLGA scaffolds can be combined together to form the composition of the present invention.

Experiment 2: Surgical Procedures in Osteochondral Model

All surgical procedures were approved by the Animal Care and Use Committee of National Cheng Kung University. Thirty-eight 4-5-month-old New Zealand White male rabbits weighing 2-3 kg were used in this study. Before surgery, anesthesia was induced with a subcutaneous injection of Zoletil 50 (25 mg/kg), followed by intubation and maintenance with a mixture of 2% isoflurane and oxygen/nitrous oxide (1/0.4 L/min) through an automatic ventilator. Under anesthesia, both legs were then shaved, brushed, disinfected with 1% ethanol-iodine, and covered with a drape. The knee was exposed by an anteromedial parapatellar longitudinal and capsular incision. The knee joint was then immobilized in the maximally hyper-flexed position. The patella was dislocated laterally to expose the medial femoral condyle. Therefore, a full-thickness osteochondral defect that was 3 mm in depth and 3 mm in diameter was created with an electric drill on the weight-bearing zone of the medial femoral condyle.

The joint was then irrigated immediately with sterile isotonic saline. After removing the debris from the defect with a curette and cleaning the defect edge with a scalpel blade, the rabbits were allocated randomly into empty defect (ED), PLGA-implanted (PI), and EPC-PLGA groups. A PLGA scaffold that was pre-sterilized in 75% ethanol was inserted gently into the defect hole by press-fit fixation and subsequently flushed with normal saline and repositioned in the patellar position, followed by wound closure. The capsule was closed carefully using 3-0 absorbable Vicryl sutures. The subcutaneous tissues and skin were repaired using 3-0 nylon sutures.

All of the rabbits were housed singly in a stainless-steel cage. An antibiotic (25 mg/kg, Enrofloxacin) and analgesic (Ketoprofene) therapy was administered immediately after each surgery and for 3 days thereafter, and the wounds were dressed with povidone-iodine for 7 days. In addition, in the sham group, no defects were created in the rabbit's knees; the knees were only opened, and the wounds were closed as described above. All of the rabbits were observed for body weight, appetite, wound healing, and proper functional activity after surgery. The rabbits were euthanized 4 or 12 weeks after surgery with an intravenous injection of 120 mg/kg pentobarbital. The result is referred to FIG. 4 to FIG. 6.

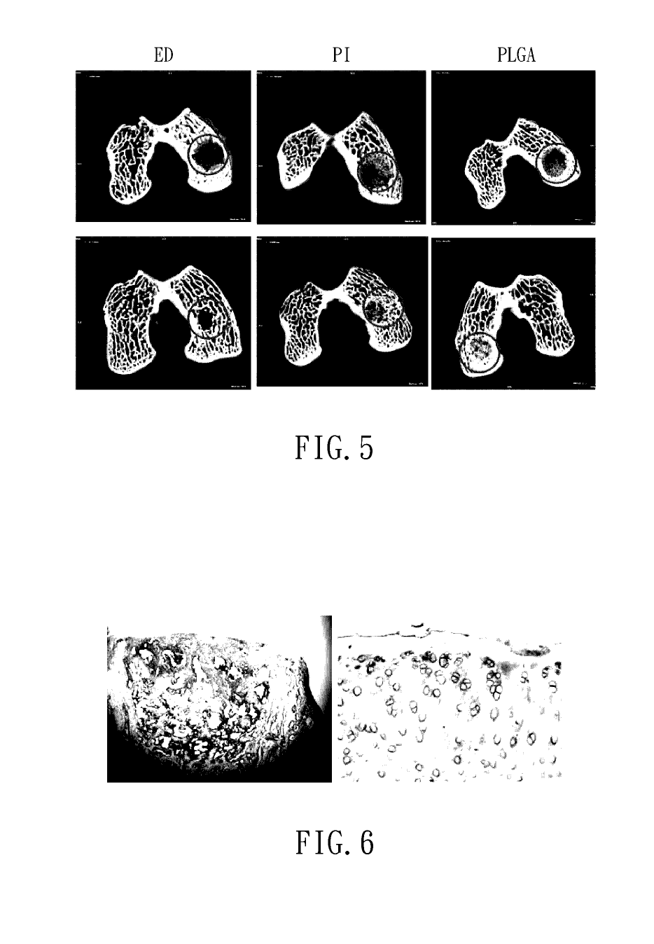

FIG. 4 is the outside view of the cartilage tissue repaired by the methods of the present invention. FIG. 5 is the image of the cartilage tissue repaired by the methods of the present invention observed by micro computed tomography (microCT). With reference to FIG. 4 and FIG. 5, the upper and the bottom row of the images were the fourth week result and the twelfth week result, respectively. From left to right were empty defect (ED) group, PLGA-implanted (PI) group, and EPC-PLGA group. As the image shown, Compared the 3 groups of fourth week condition, the repairing effect of the EPC-PLGA group is much more obvious to the other two groups. For EPC-PLGA group, the damaged area of the cartilage tissues presented better repairing effect. According to the twelfth week result, the effect of the EPC-PLGA group is much more obvious.

FIG. 6 is the result of the type II collagen included by the repaired cartilage tissue observed by immuno-histochemistry stain. With reference to FIG. 6, the left one and the right one individually shows the result of the fourth week and the twelfth week. As the image shown, the type II collagen included by repaired cartilage tissue is as predicted by the present invention. Moreover, the result of twelfth week is much more obvious than the fourth week.

It can be seen that the method of the present invention is able to effectively repair the damaged cartilage tissues, and its effect is much more obvious with the addition of the repairing day.

As mentioned above, the composition for repairing cartilage tissues provided by the present invention comprises the endothelial progenitor cells and the cell scaffold materials. Because of the simple way to harvest the endothelial progenitor cells by drawing blood, this method is able to ease the burden of invasive surgery and the pain from drawing marrow in the past. In addition, the composition composed of the endothelial progenitor cells is advantageous for short culturing time and the small amount of cell demand. This is able to shorten the treatment of cartilage repairing.

Furthermore, the composition composed of the endothelial cells and the biological scaffold and the method making the same has the following advantages. First, the endothelial progenitor cells are taken from the patient himself to prevent from the infection of the immune rejection in the allograft or the xenograft. Second, the composition is able to induce the cartilage tissues and its peripheral bone tissues to grow and repair; furthermore, forming hyaline cartilage is able to raise the effect of repairing and to recover the whole joint function of patients. Compared to the prior techniques, the composition and the method making the same provided by the present invention is advantageous for decreasing the surgery times, shortening the treatment and achieving the efficacies of fast and effectively repairing the cartilage tissues.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A composition for repairing cartilage tissues comprising:
a scaffold; and
a plurality of endothelial cells adhering on the scaffold.

2. The composition according to claim 1, wherein the scaffold is bio-compatible substance.

3. The composition according to claim 1, wherein the material of the scaffold comprises polycaprolactone (PCL), polylactic acid (PLA), polyglycolic acid (PGA), poly(lactic-co-glycolic) acid (PLGA), poly(p-dioxanone), polyanhydride, polyethylene terephthalate (PET), polyorthoester (POE), collagen, gelatin, hyaluronic acid, chitosan, or polyethylene glycol (PEG).

4. The composition according to claim 1, wherein the scaffold is multi-porous.

5. The composition according to claim 1, wherein the endothelial progenitor cells are taken from the blood of an individual having cartilage tissues to be repaired.

6. The composition according to claim 1, wherein the composition is an implant.

7. The composition according to claim 6, wherein the composition is implanted into the adjacency between the cartilage tissue and its peripheral bone tissue of an individual.

8. The composition according to claim 1, wherein the endothelial progenitor cells induce the cartilage tissues and its peripheral bone tissue to grow and repair.

9. A method of making a composition for repairing cartilage tissue comprising the following steps of:
providing a scaffold;
implanting a plurality of endothelial progenitor cells on the scaffold; and
culturing the endothelial progenitor cells on the scaffold.

10. The method according to claim 9, wherein the scaffold is bio-compatible substance.

11. The method according to claim 9, wherein the material of the scaffold comprises polycaprolactone (PCL), polylactic acid (PLA), polyglycolic acid (PGA), poly(lactic-co-glycolic) acid (PLGA), poly(p-dioxanone), polyanhydride, polyethylene terephthalate (PET), polyorthoester (POE), collagen, gelatin, hyaluronic acid, chitosan, or polyethylene glycol (PEG).

12. The method according to claim 9, wherein the scaffold is multi-porous.

13. The method according to claim 9, wherein the endothelial progenitor cells are taken from the blood of an individual having cartilage tissues to be repaired.

14. The method according to claim 9, wherein the composition is an implant.

15. The method according to claim 14, wherein the composition is implanted into the adjacency between the cartilage tissue and its peripheral bone tissue of an individual.

16. The method according to claim 9, wherein the endothelial progenitor cells induce the cartilage tissues and its peripheral bone tissue to grow and repair.

17. The method according to claim 9, wherein the endothelial progenitor cells are cultured in vitro within one week before implanting into the scaffold.

18. The method according to claim 9, wherein the endothelial progenitor cells are cultured on the scaffold within one day.
